(19) United States
(12) Patent Application Publication
(10) Pub. No.: US 2015/0148873 A1
(43) Pub. Date: May 28, 2015

(54) Therapeutic Warming Fluid

(71) Applicant: Gary Robert Prok, Rush, NY (US)

(72) Inventor: Gary Robert Prok, Rush, NY (US)

(21) Appl. No.: 14/551,456

(22) Filed: Nov. 24, 2014

Related U.S. Application Data
(60) Provisional application No. 61/963,094, filed on Nov. 22, 2013.

(51) Int. Cl. A61F 7/08; C09K 5/06

(52) U.S. Cl. CPC A61F 7/08; C09K 5/063
USPC 607/104; 252/67

(57) ABSTRACT

A solution of erythritol and water, saturated at a desired therapeutic temperature, is contained in a hot water bottle, or other appropriate vessel, for the purpose of therapeutic warming, for example, for the relief of gastro-intestinal or muscular discomfort. The heat of crystallization of the erythritol significantly extends the time of therapeutic warming. Nucleation agents are added to delay the formation of large crystals upon cooling of the solution. The resulting product is non-toxic to humans and to canines.

### CROSS-REFERENCE TO RELATED APPLICATIONS

This patent application claims the benefit of Provisional Application Ser. No. 61/963,094, THERAPEUTIC WARMING FLUID, filed Nov. 22, 2014, the disclosure of which is incorporated herein by reference in its entirety.

### BACKGROUND

1. Field of the Invention

The present invention relates in general to the use of a phase change material in a therapeutic warming device.

2. Description of the Related Art

Currently, gels and tap water are commonly used in devices for therapeutic warming. However, there remains a need for warming devices with greater heat storage capacity that deliver warmth for a longer period of time, that contain materials that are both low in cost and safe for the end user.

### SUMMARY OF THE INVENTION

In accordance with a first aspect of the invention, a therapeutic warming fluid comprising erythritol and water, is provided.

In a second aspect of the invention, a vessel for delivering therapeutic warmth containing erythritol and water, is provided.

In a third aspect of the invention, a method of delivering therapeutic warmth, comprising: contacting a patient with a vessel containing a therapeutic warming fluid comprising erythritol and water, is provided.

### DETAILED DESCRIPTION OF THE INVENTION

In accordance with an embodiment of the invention, a therapeutic warming fluid comprising erythritol and water, is provided.

In a particular embodiment, a therapeutic warming fluid comprising a saturated aqueous solution of erythritol, is provided.

In a particular embodiment, an aqueous therapeutic warming fluid comprising dissolved erythritol and crystalline erythritol, is provided.

In various embodiments, erythritol (C₄H₁₀O₄) of various isomeric forms, such as, for example, (2R,3S)-butane-1,2,3,4-tetraol (CAS No. 10030-58-7) or (2R,3R)-butane-1,2,3,4-tetraol (CAS No. 2418-52-2), also known as threitol, are employed.

Erythritol is non-toxic to humans and to canines.

In other embodiments, the erythritol is replaced, in whole or in part, by another sugar alcohol, such as, but not limited to, xylitol or pentaerythritol.

While not wishing to be bound by any particular theory, erythritol provides a large endothermic heat of dissolution, thereby providing a larger heat storage capacity when heating the therapeutic warming fluid. Correspondingly, erythritol provides a high exothermic heat of crystallization, thereby lengthening the time that the therapeutic warming fluid delivers heat.

In a particular embodiment, the therapeutic warming fluid further contains an agent capable of preventing or delaying formation of large crystals of erythritol, such as, for example, alumina, silica or nanoparticle metal oxides, stabilized for suspension in water. In a particular embodiment fumed silica is employed. In a particular embodiment, a mixture of agents capable of preventing or delaying formation of large crystals of erythritol is employed. In a particular embodiment, the agent capable of preventing or delaying formation of large crystals erythritol acts as a nucleation agent for crystallization.

A various embodiments, the therapeutic warming fluid is contained in a vessel, including rigid walled or flexible walled vessels, such as, for example, a conventional hot water bottle made of a natural or synthetic rubber. In general, any vessel suitable for delivery of therapeutic warmth may be employed.

In particular embodiments, the therapeutic warming vessel contains aqueous erythritol solutions saturated at a temperature in the range of about 120° F. to about 140° F. In general, however, the therapeutic warming vessel may be loaded with aqueous erythritol solutions saturated at any temperature tolerated by the containment vessel.

In a particular embodiment, a method of delivering therapeutic warmth, comprising: contacting a patient with a vessel containing a therapeutic warming fluid comprising erythritol and water, is provided.

In various embodiments, the temperature of the therapeutic warming fluid in contact with a patient ranges from about 80° F. to about 180° F., from about 100° F. to about 160° F., or from about 120° F. to about 140° F. In a particular embodiment, temperature of the therapeutic warming fluid in contact with a patient is about 130° F.

In various embodiments the patient to which therapeutic warmth is delivered is a human or a non-human subject, such as another mammal, including, but not limited to, a canine, a feline, a bovine, an equine, an ovine, a porcine, or a rodent. Alternatively, the patient or recipient of therapeutic warmth can be an animal such as a bird, insect, reptile, amphibian, or any companion or agricultural animal.

The invention is further illustrated by the following example, which is not intended to limit the invention in any manner.

### Experimental Section

A saturated solution of water and erythritol was formed by dissolving erythritol in water, heating the resulting solution to about 130° F., and adding additional erythritol until a saturated solution was formed. The saturated aqueous solution of erythritol was transferred to a conventional hot water bottle made of silicone rubber. The bottle was sealed and allowed to cool to room temperature, during which a portion of the dissolved erythritol formed crystalline solids. A vessel containing a mixture of a saturated aqueous solution of erythritol and precipitated erythritol solids was thus formed.

The bottle containing the therapeutic warming fluid comprising erythritol and water, prepared as described above, was heated in a microwave oven for several minutes until the temperature of the contents was raised to about 130° F., during which a large portion of the erythritol solids redissolved. It was noted that substantially more microwave energy (i.e. longer heating time) was required to raise the bottle containing the therapeutic warming fluid comprising erythritol and water to the desired temperature compared to a similar type of bottle containing only water.

The warmed bottle containing the therapeutic warming fluid comprising erythritol and water was placed in contact with the muscles of a patient and heat was transferred. It was observed that the bottle containing the therapeutic warming fluid comprising erythritol and water delivered therapeutic warmth for a substantially longer period of time compared to a similar type of bottle containing only water.

While the invention has been described by reference to various specific embodiments, it should be understood that numerous changes may be made within the spirit and scope of the inventive concepts described. Accordingly, it is intended that the invention not be limited to the described embodiments, but will have the full scope defined by the claims.

What is claimed:

1. A therapeutic warming fluid comprising erythritol and water.

2. The therapeutic warming fluid of claim 1, wherein said fluid comprises a saturated aqueous solution of erythritol.

3. The therapeutic warming fluid of claim 1, wherein said fluid comprises a mixture of dissolved erythritol and crystalline erythritol.

4. The therapeutic warming fluid of claim 1, further comprising an agent capable of preventing or delaying formation of large crystals erythritol.

5. The therapeutic warming fluid of claim 1, further comprising alumina, silica or a metal oxide nanoparticle.

6. A vessel for delivering therapeutic warmth, containing a therapeutic warming fluid comprising erythritol and water.

7. A method of delivering therapeutic warmth, comprising: contacting a patient with a vessel containing a therapeutic warming fluid comprising erythritol and water.
